(19) United States
(12) Patent Application Publication
(10) Pub. No.: US 2024/0269095 A1
(43) Pub. Date: Aug. 15, 2024

(54) Rapid Onset Therapeutic Ketosis Enabled by D-BetaHydroxyButyric Acid Systems, Compounds and Methods for Stroke medication co-administration

(71) Applicant: American Ketone Therapeutics LLC, Sheridan, WY (US)

(72) Inventor: James Schroeder, Seattle, WA (US)

(21) Appl. No.: 18/400,189

(22) Filed: Dec. 29, 2023

Related U.S. Application Data
(60) Provisional application No. 63/478,103, filed on Dec. 30, 2022.

(51) Int. Cl. A61K 31/19; A61B 5/00; A61B 5/026; A61B 5/145; A61K 45/06

(52) U.S. Cl. CPC A61K 31/19; A61B 5/026; A61B 5/14532; A61B 5/14546; A61B 5/4566; A61K 45/06

(57) ABSTRACT

The claimed uses of bioidentical free D-BetaHydroxyButyric Acid as a method (1303), system (1411) and optional co-adjuvant compound for pharmaceutical co-administration with stroke therapeutic pharmaceutical compounds to treat stroke are novel, safe and not anticipated by the body of research spanning well over 75 years. Overall, no adverse findings have been reported within cell, animal and human safety studies at high dose levels with D-BetaHydroxyButyric acid which supports the safety and efficacy of the claimed uses at the anticipated usage levels for the disclosed applications.

### CROSS-REFERENCE TO RELATED APPLICATIONS

This patent application is related to and claims priority from previously filed provisional application Ser. No. 63/478,103 filed on Dec. 30, 2022.

### TECHNICAL FIELD

The claimed invention has applicability in pharmaceutical stroke medication. With greater particularity, the claimed invention is relevant to therapeutic interventions for stroke. With still greater particularity, the claimed invention is directed at novel uses of the ketone body D-HydroxyButyric Acid for treatment of stroke together with and independently of pharmaceutical stroke medication with related systems and methods.

### BACKGROUND OF THE INVENTION

A stroke occurs when the blood supply to brain tissue is blocked by a blood clot (ischemic stroke), or when a blood vessel in the brain ruptures (hemorrhagic stroke), causing brain cells to die and leading to functional impairments. Stroke is a leading cause of death and disability both globally and in the U.S., where approximately 800,000 people experience a stroke each year. Although stroke remains a critical health issue, better management of cardiovascular risk factors, greater awareness of symptoms, and prompt medical attention are helping to prevent strokes and improve outcomes. Accordingly, the death rate from stroke in the U.S. fell 77% between 1969 and 2013. Another major advance was the clot-dissolving medicine tPA (for tissue plasminogen activator), the first treatment for acute ischemic stroke to receive Food and Drug Administration (FDA) approval.

Known by the generic name alteplase and marketed as Activase® (Genentech), tPA is given to patients through an IV in the arm, and it works by dissolving blood clots that block blood flow to the brain. When administered quickly after stroke onset (within three hours, as approved by the FDA), tPA helps to restore blood flow to brain regions affected by a stroke, thereby limiting the risk of damage and functional impairment. Tissue Plasminogen Activator for Acute Ischemic Stroke (Alteplase, Activase®) has a limited window of opportunity for administration.

Spinal cord infarction is a stroke either within the spinal cord or the arteries that supply it. It is caused by arteriosclerosis or a thickening or closing of the major arteries to the spinal cord. Frequently spinal cord infarction is caused by a specific form of arteriosclerosis. A spinal stroke—also called a spinal cord stroke—occurs when the blood supply to a section of the spinal cord is cut off. The spinal cord is part of the central nervous system (CNS), which also includes the brain. When the blood supply is cut off, the affected part of the spinal cord can't get oxygen and nutrients. The tissues may be damaged and not be able to send nerve impulses (messages) to the rest of your body. These nerve impulses are vital for controlling activities of the body, such as moving the arms and legs and allowing your organs to work properly.

The majority of spinal strokes are caused by a blockage in the blood vessels that supply blood to the spinal cord, such as a blood clot. These are called ischemic spinal strokes. A small number of spinal strokes are caused by bleeds. These are called hemorrhagic spinal strokes. A spinal stroke is different than a stroke that affects the brain. In a brain stroke, the blood supply to a part of the brain decreases. Spinal strokes are much less common than strokes that affect the brain, accounting for no more than 1 percent of all strokes. Spinal stroke treatment is aimed at treating the cause of the spinal stroke and reducing symptoms, and include antiplatelet and anticoagulant drugs, such as heparin and warfarin (Coumadin).

### BRIEF SUMMARY OF THE INVENTION

Overview of Disclosure: The novel benefits of D-Beta HydroxyButyric Acid arise from exogenous bolus administration in chemical form. The first section of the disclosure addresses the enabling benefits of exogenous D-Beta HydroxyButyric Acid administered in the absence of a chemical salt or ester configuration, followed by an identity section further detailing chemical identity of D-BHB followed by blood level ketosis data according to the claimed invention.

Introduction: D-BetaHydroxyButyric acid (D-BHB) reduces inflammation, improves atherosclerosis along with improving brain energy. All of these attributes increase opportunities for improved outcomes when combined with medically approved use of stroke related pharmaceuticals including Tissue Plasminogen Activator (TPA) for Acute Ischemic Stroke (Alteplase, Activase®) and for spinal stroke. Free D-BHB acid is superior to commonly known ketone salts and ketone esters owing to its rapid bioavailability and absence of chemical salt or ester bond. Free D-BHB acid also has an opportunity for stroke improvement in the absence of co-administered pharmaceuticals as well. D-BHB is direct Mitochondrial Brain Energy & Anti-Inflammation which synergistically creates enhanced recovery owing to improved mitochondrial bioenergenics during stroke. With enhanced mitochondrial energy, brain tissues at risk of dying improve opportunities for recovery for the brain cell being challenged, for the neural tissue at risk of impairment and for the individual in terms of healthy outcome vs impairment or death.

The ketogenic diet and more recently administration of ketogenic compounds have had a similar long history with proven benefits for conditions such as epilepsy. Dietary interventions for ketogenic diet treatment of atherosclerosis are yielding compelling results. The administration of the natural form of D-BetaHydroxyButyric Acid as an exogenous compound has rapid onset and direct benefits to brain bioenergenics and blood circulation dynamics. Preliminary data indicates that individuals suffering from atherosclerosis often benefit from administration of D-BetaHydroxyButyric Acid in the absence of a salt or ester.

The claimed invention combines these two physiological approaches for stroke intervention into a combinatorial delivery system for improved physiological outcomes in the treatment of stroke related physiological conditions. The exogenous ketone D-BHB Acid is either co-administered with a stroke pharmaceutical compound or subsequently administered with the following expected benefits:

Firstly, with the introduction of D-BetaHydroxyButyric Acid improving brain bioenergenics and reducing neuro-inflammation, it is a foreseeable consequence of the claimed invention that a broader window of administration of pharmaceuticals such as tPA, Alteplase and/or TNKase® Tenecteplase will be increased leading to a higher percentage of positive physiological recovery breakthroughs. Secondly, the present form of D-BetaHydroxyButyric Acid is rapidly absorbed and immediately bioavailable which is in direct contrast to the moderately ketogenic ketone salts and ketone ester compounds which require stomach disassociation followed by liver processing. It is believed that the greatly improved bioavailability of free D-BHB acid will improve therapeutic outcomes for stroke both in conjunction with current standard of care pharmaceuticals as well as when administered alone subsequent to stroke events or even as a prophylactic.

TABLE 1
Free D-β-HydroxyButyric Acid (D-BHB)
Free D-BHB is both mitochondrial energy source
and regulatory compound with the fastest
bioavailability and efficacy vs synthetic ester
and chemical salt.
Unexpected cognitive bioenergenics and
neuroprotection benefits.
Free D-BHB is a rapid release format.
Free D-BHB is safe and non-toxic.
Effects: Regulatory, Direct Mitochondrial Energy
Metabolism: Rapid, minutes
Use in Humans: Ancient
Induces Ketoacidosis: No

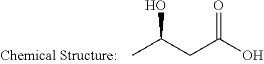

The rationale for a bioidentical D-BHB therapeutic regime is primarily supported by the neuroinflammation reduction properties of bioidentical D-BHB, high ketone levels generated by the same coupled with high bioavailability without salt load or liver metabolism requirements. As the bioidentical ‘free’ form. The bioidentical ‘free’ D-BHB form is immediately bioavailable for rapid metabolism which is materially distinct from the ‘ketone salt’ compounds (often D+L racemic) chemically joined to a salt or the synthetic ‘ketone ester’ compounds which are alcohol based and must be processed in the stomach and liver before releasing the ketone contained therein.

### BRIEF DESCRIPTION OF THE SEVERAL VIEWS OF THE DRAWING(S)

The accompanying drawings are included to better illustrate exemplary embodiments of the claimed invention.

FIG. 1 is a structural illustration of endogenous ketones.

FIG. 2 is a structural illustration of chemical structures of bioavailable free D-BetaHydroxyButyric Acid, ketone ester and ketone salt.

FIG. 3 is a graphical illustration of endogenous ketone levels by age.

FIG. 4 is a graphical illustration of endogenous ketone brain metabolism.

FIG. 5 is a graphical illustration of comparative exogenous ketone blood levels.

FIG. 6 is a graphical illustration of brain metabolism of ketones.

FIG. 7 is a graphical illustration of free D-BetaHydroxyButyric Acid preparation for stroke administration.

FIG. 8 is a graphical illustration of free D-BetaHydroxyButyric Acid preparation for spinal ischemia administration.

FIG. 9 is a graphical illustration of D-BHB exogenous ketone blood levels.

FIG. 10 is a graphical illustration of D-BHB exogenous ketone blood levels.

FIG. 11 is a graphical illustration of continuous monitoring of body glucose levels.

FIG. 12 is a graphical illustration of continuous monitoring of body ketone levels resulting in improved deep sleep time due to enhanced D-BHB levels according to the claimed invention.

FIG. 13 is a schematic illustration of a preferred D-BHB stroke management method with optional stroke therapeutic pharmaceutical administration.

FIG. 14 is a schematic illustration of a preferred D-BHB stroke management system with optional stroke therapeutic pharmaceutical administration.

### DETAILED DESCRIPTION OF THE INVENTION

Introduction: The claimed invention alleviates stroke by way of two primary embodiments. The first primary embodiment addresses stroke by reducing neuro-inflammation and increasing direct brain energy by exogenously administering bioidentical D-BetaHydroxyButyric Acid. The second primary embodiment improves stroke therapeutic pharmaceuticals by co-administration of D-BetaHydroxyButyric Acid to optimize stroke therapeutic pharmaceutical compounds.

### I. D-Beta HydroxyButyric Acid Compound Disclosure and Stroke Applications.

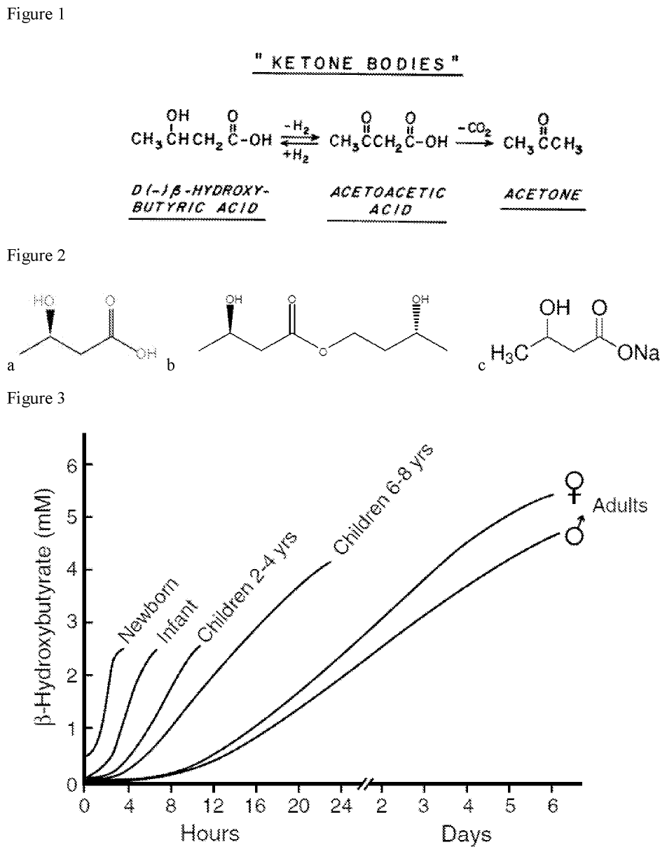

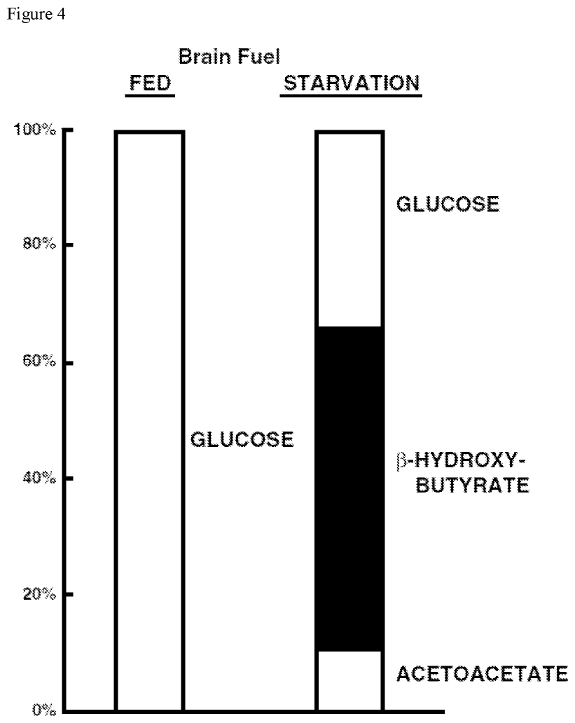

FIG. 1 is a structural illustration of endogenous ketones. The illustration of the endogenously produced ketone bodies is from FIG. 4 of the Harvard Medical School Professor/Dr. George F. Cahill, Jr.'s lecture entitled “Ketosis” given over fifty years ago. Dr. Cahill's foundational work provides clear and concise understanding of ketosis to aid in the understanding of the claimed invention. The FIG. 2 (a) chemical formula identifies the claimed bioidentical ‘free’ form of the exogenous D-Beta HydroxyButyric Acid administered in the absence of a chemical salt or ester and is the subject of ClinicalTrials.gov identifier NCT05584371. The claimed exogenously administered bioidentical free D-BetaHydroxyButyric Acid is chemically identical to the D-BetaHydroxyButyric Acid identified in FIG. 1. Greater detail is hereby provided in the submitted BMJ Nutrition, Prevention & Health journal article. To be clear, this publication is the first reported clinical trial of bioidentical free D-BetaHydroxyButyric acid administered exogenously. The intervening fifty years between the Jeremiah Metzger Lecture given in Boston in 1972 and the present clinical trial and disclosure do not detail bioidentical free D-BetaHydroxyButyric Acid but instead disclose a wide variety of precursors, chemical combinations and not naturally occurring synthetic products which are not bioidentical in their administered form.

FIG. 2 is a structural illustration of bioidentical ketone and chemically synthetic chemical ketone structures. FIG. 2(a) depicts bioidentical free BetaHydroxyButyric Acid. FIG. 2(b) depicts the synthetic ketone ester and FIG. 2 (c) shows the chemical ketone salt. Put simply, bioidentical free D-Beta HydroxyButyric Acid offers direct mitochondrial brain energy resulting in optimized therapeutic outcomes when administered in the absence of a chemical salt or ester configuration. To date, exogenous ketone supplementation has been attempted as an aide in following the ketogenic diet. Unfortunately, ketone salts result in an abnormally high salt intake rate and the newly created (R)-3-hydroxybutyl (R)-3-hydroxybutyrate (D-β-hydroxybutyrate ester) or ‘ketone ester’ is a never before seen in man synthetic creation with bioavailability and other long-term concerns. FIG. 2(a) illustration of the chemical form of bioidentical free D-BetaHydroxyButyric Acid is also distinguishable from ketone salts and ketone esters owing to its immediate bioavailability and absence of chemically joined salt or ester bond.

FIG. 3 is a graphical illustration of endogenous ketone levels by age and FIG. 4 is a graphical illustration of endogenous ketone brain metabolism from Dr./Prof. Cahill's journal article “Fuel Metabolism in Starvation” provided by Information Disclosure Statement. FIG. 3 details the time to ketosis in healthy adults vs children measured in hours or days. D-BetaHydroxyButyric Acid is a naturally occurring ketone which is produced by the human body. The ketone plays a crucial developmental role during development and throughout the healthy human lifespan. It is produced naturally during fasting (detailed by Cahill's graph above) as well as through the use of ketogenic diets. As Cahill observed, “Due to its use by brain, D-β-HydroxyButyric acid not only has permitted man to survive prolonged starvation, but also may have therapeutic potential owing to its greater efficiency in providing cellular energy in ischemic states such as stroke, myocardial insufficiency, neonatal stress, genetic mitochondrial problems, and physical fatigue.” Cahill's early insights on D-β-HydroxyButyric acid are now through Applicant's clinical trial efforts becoming proven in reality. Applicant's clinical trials including ClinicalTrials.gov identifier NCT05584371 are the first documented clinical applications of free D-BetaHydroxyButyric Acid as further detailed in the provided Dr. Prof. Soto-Mota IDS journal article. FIG. 4 details the fact that ketones in general, and D-BetaHydroxyButyric Acid in particular is the preferred fuel in the human brain. The time to ketosis levels are relevant in the context of the instant application in that creating a rapid 1.5-2.5 mmol/L increase in ketone levels is simply not practical through fasting or diet when rapidly needed in a stroke context.

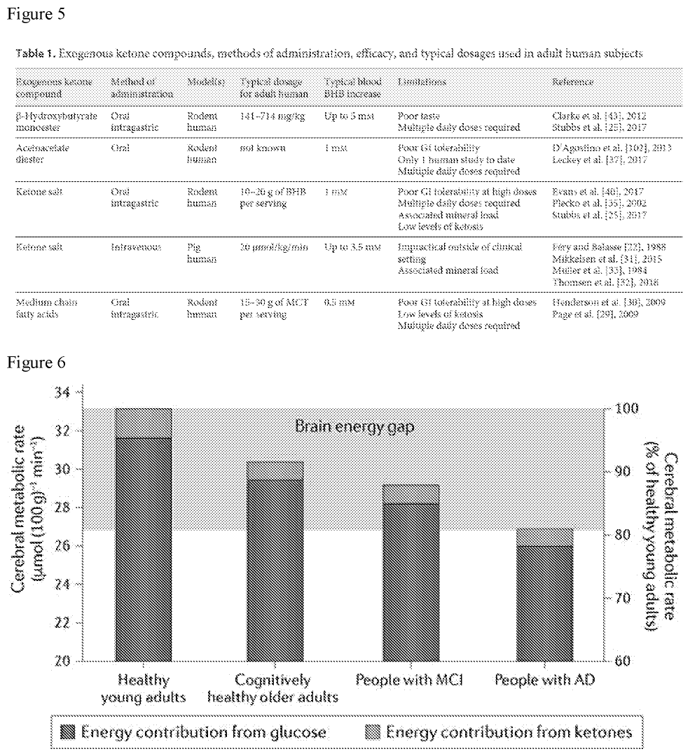

FIG. 5 is a graphical illustration of comparative exogenous ketone blood levels. FIG. 5 depicts Table 1 from “Exogenous Ketone Bodies as Promising Neuroprotective Agents for Developmental Brain Injury” and details the significant limitations among the variety of previously known exogenous salt and ester ketone compounds. Ketone salts are often racemic D+L mixtures which rarely provide greater than 1 mmol/L ketone increase along with substantial salt load rendering it unsuitable for long term daily administration. Similarly, while the synthetic ‘ketone ester’ does provide a higher ketone level response, its highly unpalatable taste renders it highly unsuitable for use in mental health applications. FIG. 5 does not include free D-BetaHydroxyButyric Acid due to the fact that ClinicalTrials.gov identifier NCT05584371 and the provided BMJ Nutrition, Prevention & Health journal article is the first instance of exogenous free D-BetaHydroxyButyric Acid journal publication. Put simply, there has been a longstanding need for a salt free, bioidentical solution to rapid ketosis without alcohol or need for further liver metabolism to rapidly increase ketones to a level relevant for mental health protection and recovery.

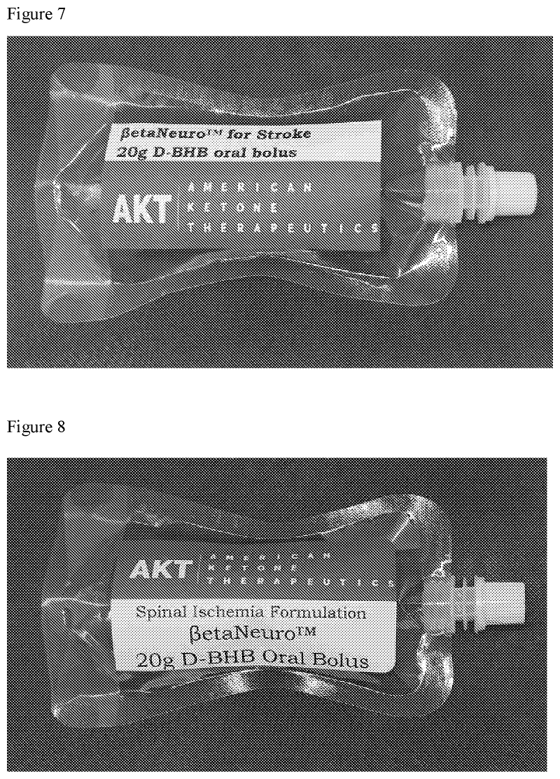

FIG. 6 is a graphical illustration of brain metabolism of ketones from Professor Stephen Cunnane's journal article “Brain energy rescue: an emerging therapeutic concept for neurodegenerative disorders of ageing” figure number 2. This figure details “Causes and consequences of the brain energy gap in neurodegenerative disorders . . . Glucose contributes to about 95% of total brain fuel supply in cognitively healthy young adults, and ketones supply the remaining 5%. In cognitively healthy older adults, brain glucose uptake is decreased by about 9%, in people with mild cognitive impairment (MCI) it is decreased by about 12% and in people with mild-to-moderate AD it is decreased by about 18%. The magnitude of the brain energy gap is the difference in total brain fuel uptake (glucose and ketones combined) between healthy young adults and people with mild-to-moderate AD; that is, the therapeutic target for brain energy rescue in MCI and AD. The brain energy gap has not been rigorously quantified in neurodegenerative disorders of ageing other than AD.” Applicants respectfully submits that Professor Cunnane's ‘brain energy gap’ model for mild cognitive impairment may be a useful framework and helpful analogy when addressing symptoms of stroke related disorders and a similar gap spectrum may exist when considering the differences between healthy individuals and individuals suffering from stroke. As supplementation with bioidentical free D-BetaHydroxyButyric Acid is direct mitochondrial brain energy it is the most suitable compound for restoration and closure of the ‘brain energy gap’ in individuals suffering from a stroke event. It is a direct and foreseeable consequence of the claimed invention that similar benefits against stroke and spinal ischemia are achieved. As an optional anticipated embodiment, utilization of exogenous free D-BetaHydroxyButyric Acid to compensate for the brain energy gap deficiency will yield higher positive outcomes when utilizing stroke therapeutic pharmaceuticals.

FIG. 7 is a graphical illustration of free D-BetaHydroxyButyric Acid preparation for stroke administration. Many stroke patients arrive at the hospital intubated for rapid oral pharmaceutical administration. The depicted preparation of D-BHB free acid is designed for rapid and easy intubated administration. While 20 g D-BHB free acid is depicted a wide range of D-BHB administration is anticipated according to the claimed invention. Often the stroke patient has suffered a stroke at night which is not detected until the following morning. Such uncertainty around the time of cerebral injury or insult greatly restricts the window of efficacy of pharmaceuticals such as tPA. Rapid administration of D-BHB free acid will provide enhanced brain bioenergenics to increase the window of tPA efficacy and improve stroke recovery.

FIG. 8 is a graphical illustration of free D-BetaHydroxyButyric Acid preparation for spinal ischemia administration. Spinal ischemia patients often arrive at the hospital intubated for rapid oral pharmaceutical administration especially when subject to substantial physical trauma or accident. The depicted preparation of D-BHB free acid is designed for rapid and easy intubated administration. While 20 g D-BHB free acid is depicted a wide range of D-BHB administration in grams is anticipated according to the claimed invention. In addition to the aforementioned bioenergetic properties, long term administration of D-BHB free acid improves spinal ischemia prognosis owing to the enhanced atherosclerosis attributes even in the absence of a co-administered pharmaceutical. It is an anticipated embodiment of the claimed invention that in addition to exigent D-BHB administration, follow-on D-BHB administration between 5 g and 30 g regularly administered over days, weeks, months and even years can improve brain stroke and spinal ischemia positive outcomes and enhance recovery.

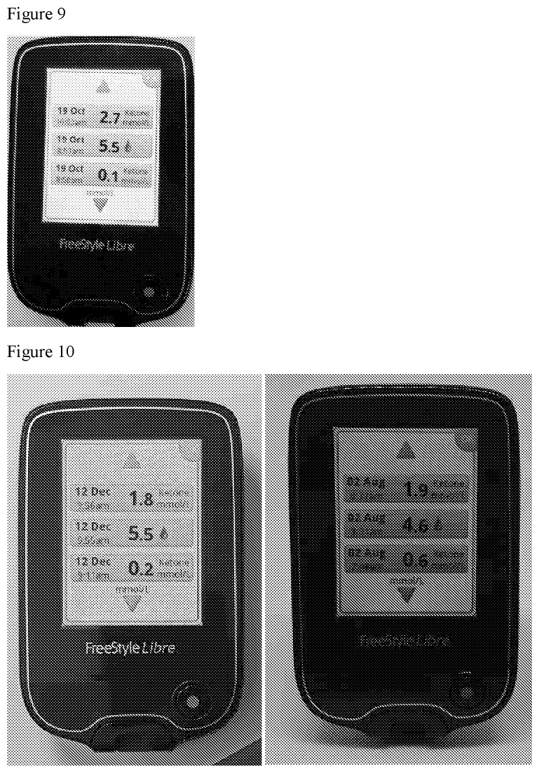

FIG. 9 is a graphical illustration of D-BHB exogenous ketone blood levels. The above Abbot blood ketone meter evidenced by Abbott blood test strip depicts the rapid rise in blood ketone levels from 0.1 mmol/L (absence of ketosis) to 2.7 mmol/L (ketosis) after oral bolus administration of 15 grams of bioidentical free D-BetaHydroxyButyric Acid. The ketone levels depicted are attained in the absence of a salt as well as without ester bonds. It is the free acid D-BHB exogenously administered which is bio-identical to the endogenous D-BHB free acid generated by the human body. The bioidentical ketone body D-BHB is first and foremost an energy source, which is directly and preferentially metabolized by the brain. D-BHB is also active as a signaling mechanism and neuro-inflammation reduction agent, resulting in broad and beneficial brain bioenergy related benefits.

FIG. 10 is a graphical illustration of D-BHB exogenous ketone blood levels. FIG. 10 details rapid ketosis within 45 minutes confirmed by Abbott blood test strip after 10 g D-BHB with palatability agent administered by oral drink. Blood ketone levels document rise from 0.2 mmol/L to 1.8 mmol/L and 0.6 mmol/L to 1.9 mmol/L in a variety of test subjects. FIG. 10 reinforces the fact that supplementation by utilizing free D only BetaHydroxyButyric Acid without ketone salt or ketone ester is a reliable, reproducible method and system for rapid ketone level increase for stroke recovery and maintenance. While amounts of free D only BetaHydroxyButyric Acid taken for stroke effects may vary, it is an anticipated preferred embodiment that levels between 5 and 30 grams of D only BetaHydroxyButyric Acid are taken as needed for optimum recovery. Alternate embodiments may include lower or higher amounts, however, experimental evidence has confirmed optimal administration in this range for a wide variety of other applications.

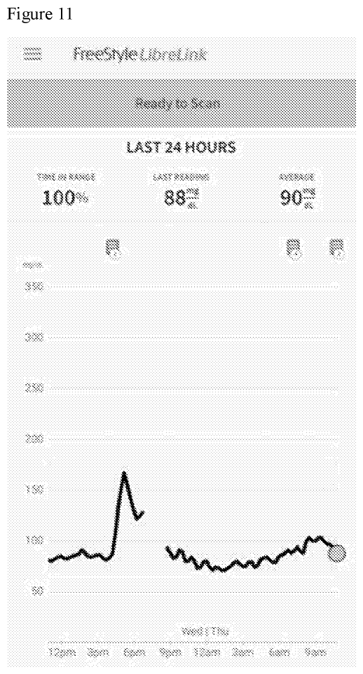

FIG. 11 is a graphical illustration of continuous monitoring of body glucose levels. FIG. 11 depicts continuous glucose monitoring levels with carbohydrate challenge documented by Abbot Libre Continuous Glucose Monitoring (CGM) device. In this illustrative embodiment, glucose levels from a 50 g carbohydrate ingestion are depicted unaided by free D-BetaHydroxyButyric Acid on the left and repeated the following day with two 50 g carbohydrate ingestions accompanied by 10 g free D-BetaHydroxyButyric Acid administered 45 minutes before the carbohydrate insult. The data is noteworthy in that while the equivalent amount of identical carbohydrates were consumed, the rapid glucose spike depicted in the unsupplemented ingestion is not found with 10 g D-BetaHyrdoxyButyric Acid accompaniment. This data is relevant in the stroke context owing to the fact that certain individuals suffering from brain energy deficits often report higher susceptibility due to low ketones and high glucose. As free D-BetaHydroxyButyric Acid can lower glucose while rapidly increasing ketone levels, it is believed that obtaining high ketones while lowering glucose will yield additional stroke recovery benefits for high glucose sensitive individuals suffering from stroke.

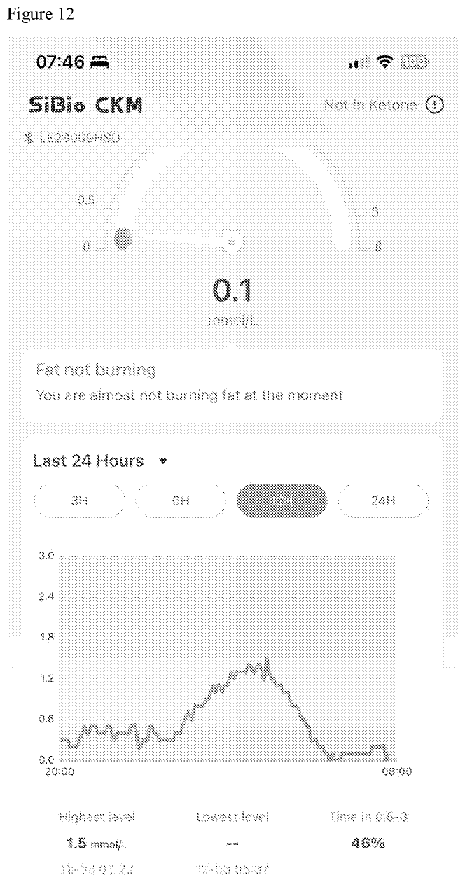

FIG. 12 is a graphical illustration of continuous monitoring of body ketone levels resulting in improved deep sleep time due to enhanced D-BHB levels according to the claimed invention. It is noteworthy that consumption of even small amounts of free D-BetaHydroxy Butyric Acid can give rise to therapeutic ketosis levels as measured by Continuous Ketone Meter (CKM). In this representative example, 5 grams of free D-BetaHydroxyButyric Acid consumed 45 minutes prior to bedtime resulted in a 1.5 mmol/L increase can also enhance the quantity of deep sleep obtained. While free D-BetaHydroxyButyric Acid is not a sedative, even small amounts can greatly improve deep sleep quantity and quality. It is an intended and foreseeable embodiment application to increase deep sleep with free D-BetaHydroxyButyric Acid with the aim of improving stroke recovery and enhanced brain resiliency.

### II. D-Beta HydroxyButyric Acid Stroke Recovery Method & System Disclosure and Applications for Pharmaceutical Co-Administration

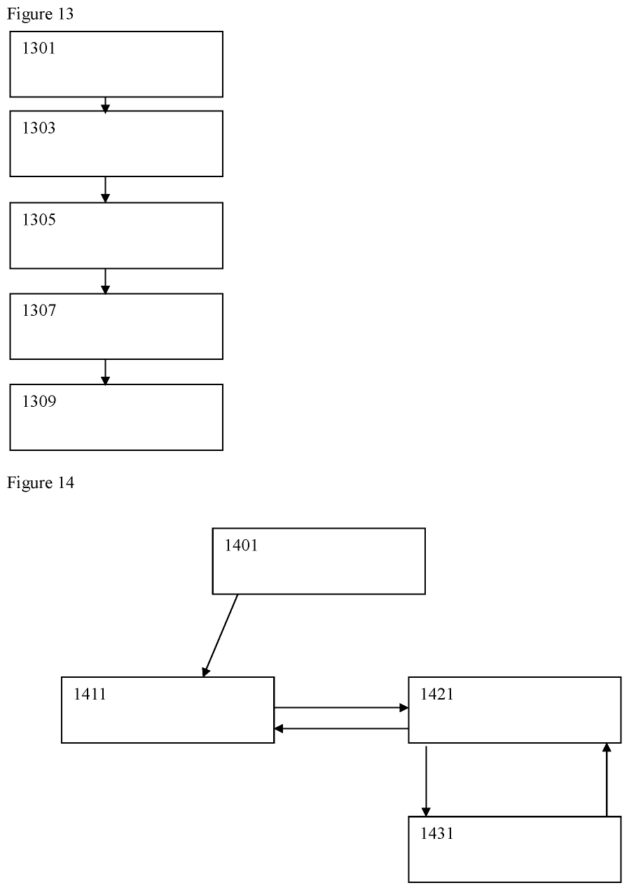

FIG. 13 is a schematic illustration of a preferred D-BHB stroke management method with optional pharmaceutical administration.

In a preferred embodiment, a method of stroke symptom management is obtained through the steps of:
Assessing (1301) ketone levels and stroke symptoms,
Administering (1303) a therapeutic amount of free D-BetaHydroxyButyric acid,
Optionally dosing (1305) a therapeutic amount of stroke therapeutic pharmaceutical,
Evaluating (1307) the depressive symptoms of a subject in need, and
Readministering (1309) free D-BetaHydroxyButyric acid alone or in combination with optionally redosing together with a therapeutic amount of stroke therapeutic pharmaceutical.

FIG. 14 is a schematic illustration of a preferred D-BHB stroke management system with optional stroke therapeutic pharmaceutical administration. In the illustrative embodiment, ketone level monitor (1421) determines the amount of free D-BetaHydroxyButyric Acid for a stroke subject in need thereof, optionally and additionally informed by glucose monitor (1433). Free D-BetaHydroxyButyric Acid therapeutic dose (1411) is administered alone or in conjunction with optional stroke therapeutic pharmaceutical therapeutic dose (1401).

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments. In particular, free D-BetaHydroxyButyric Acid may be administered diluted or with a preferred palatability agent without detracting from the spirit or scope of the claimed invention.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

I claim:

1. A method of stroke symptom management comprising the steps of:
Assessing ketone levels and stroke symptoms,
Administering a therapeutic amount of free D-BetaHydroxyButyric acid,
Evaluating the stroke symptoms of a subject in need, and
Readministering free D-BetaHydroxyButyric acid until stroke symptoms are reduced.

2. The method of claim 1 wherein said Administering a therapeutic amount of free D-BetaHydroxyButyric Acid is provided between 5 grams and 30 grams of free D-BetaHydroxyButyric Acid.

3. The method of claim 2 wherein the Assessing ketone levels and stroke symptoms further comprise assessing spinal ischemia symptoms.

4. The method of claim 1 additionally comprising dosing a therapeutic amount of stroke related therapeutic substance as a co-adjuvant simultaneously delivered with Administering a therapeutic amount of Free D-BetaHydroxyButyric Acid.

5. The method of claim 1 additionally comprising dosing a therapeutic amount of stroke related therapeutic substance as a co-adjuvant delivered more than 15 minutes after Administering a therapeutic amount of Free D-BetaHydroxyButyric Acid.

6. The method of claim 1 additionally comprising dosing a therapeutic amount of stroke related therapeutic substance as a co-adjuvant delivered more than 15 minutes before Administering a therapeutic amount of Free D-BetaHydroxyButyric Acid.

7. The method of claim 6 additionally comprising Readministering free D-BetaHydroxyButyric Acid.

8. The method of claim 7 wherein the Readministering free D-BetaHydroxyButyric Acid wherein the Readministering free D-BetaHydroxyButyric Acid is performed daily until stroke symptoms are substantially mitigated.

9. The method of claim 6 wherein the stroke related therapeutic substance is selected from the group consisting of Tissue Plasminogen Activator (TPA), Alteplase, Tenecteplase and antiplatelet and anticoagulant drugs, heparin and warfarin administered in a physiologically therapeutic amount.

10. The method of claim 9 additionally comprising Readministering free D-BetaHydroxyButyric Acid.

11. A stroke management system comprising a ketone level monitor which determines the amount of free D-BetaHydroxyButyric Acid for a stroke subject in need thereof, a glucose monitor and Free D-BetaHydroxyButyric Acid therapeutic dose.

12. The stroke management system of claim 11 wherein the amount of free D-BetaHydroxyButyric Acid administered is between 5 grams and 30 grams of free D-BetaHydroxyButyric acid.

13. The system of claim 12 wherein the ketone level monitor which determines the amount of free D-BetaHydroxyButyric Acid for a stroke subject in need thereof additionally comprises assessing spinal ischemia symptoms.

14. The stroke management system of claim 12 additionally comprising a stroke related therapeutic substance selected from the group consisting of Tissue Plasminogen Activator (TPA), Alteplase, Tenecteplase and antiplatelet and anticoagulant drugs, heparin and warfarin administered in a physiologically therapeutic amount.

15. The system of claim 11 wherein the ketone level monitor which determines the amount of free D-BetaHydroxyButyric Acid for a stroke subject in need thereof additionally comprises functionality for daily delivery of free D-BetaHydroxyButyric Acid until stroke symptoms are substantially mitigated.

16. A therapeutic compound comprising D-BetaHydroxyButyric Acid administered in 5 g-30 g format in conjunction with a stroke related therapeutic substance selected from the group consisting of Tissue Plasminogen Activator (TPA), Alteplase, Tenecteplase and antiplatelet and anticoagulant drugs, heparin and warfarin administered in a physiologically therapeutic amount.
